(19) United States
(12) Patent Application Publication
(10) Pub. No.: US 2008/0097204 A1
(43) Pub. Date: Apr. 24, 2008

(54) Method and System for Generating 3D Ultrasound Image Data and Viewing Media

(75) Inventors: Karl E. Thiele, Andover, MA (US); Mary Kay Bianchi, Plaistow, NH (US); Hugo Matthieu Visser, Utrecht (NL); Murray Fulton Gillies, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/572,649

(22) PCT Filed: Jul. 21, 2005
(86) PCT No.: PCT/IB05/52448

§ 371 Date: Jan. 25, 2007

Related U.S. Application Data
(60) Provisional application No. 60/591,073, filed on Jul. 26, 2004.

(51) Int. Cl. A61B 8/00

(52) U.S. Cl. 600/437

(57) ABSTRACT

According to one embodiment of the present disclosure, a method of providing 3D data sets for stand-alone use includes providing a 3D viewing program on a computer readable media and providing at least one 3D data set on the same computer readable media, wherein responsive to a computer activation of the 3D viewing program, the 3D viewing program operates to provide a 3D rendering based upon the at least one 3D data set.

The present disclosure generally relates to ultrasound devices, and more particularly, to a method and system for generating 3D ultrasound image data and viewing media.

Ultrasound imaging has been widely used to observe tissue structures within a human body, such as the heart structures, the abdominal organs, the fetus, and the vascular system. Ultrasound imaging systems include a transducer array connected to multiple channel transmit and receive beamformers applying electrical pulses to the individual transducers in a predetermined timing sequence to generate transmit beams that propagate in predetermined directions from the array.

As the transmit beams pass through the body, portions of the acoustic energy are reflected back to the transducer array from tissue structures having different acoustic characteristics. The receive transducers (which may be the transmit transducers operating in the receive mode) convert the reflected pressure pulses into corresponding RF signals that are provided to the receive beamformer. Due to different distances to the individual transducers, the reflected sound waves arrive at the individual transducers at different times, and thus the RF signals have different phases.

The receive beamformer has a plurality of processing channels with compensating delay elements connected to a summer. The receive beamformer uses a delay value for each channel and collect echoes reflected from a selected focal point. Consequently, when delayed signals are summed, a strong signal is produced from signals corresponding to this point, but signals arriving from different points, corresponding to different times, have random phase relationships and thus destructively interfere. Furthermore, the beamformer selects the relative delays that control the orientation of the receive beam with respect to the transducer array. Thus, the receive beamformer can dynamically steer the receive beams that have desired orientations and focus them at desired depths. In this way, the ultrasound system acquires echo data.

Presently, when patients go for an obstetrical ultrasound exam, the patient often receives a 2D photo image of her baby in utero. In addition, over the past 10 years, ultrasound has advanced, such that 3D scans of the fetus are now quite common. However, even though the scan is in 3D, the patient still receives a simple 2D photo image of her baby.

In addition, should an attending physician decide to obtain a second opinion on a 3D ultrasound image, the attending physician would likely forward a 2D picture, video tape, or “x-ray” to a second doctor. In such a situation, it may be highly likely that the second doctor does not have the necessary software to review the 3D data set in its native format. One reason for the second doctor not having the necessary software to review the 3D data sets might be that current 3D viewing and rendering software costs on the order of between $10,000 to $30,000. Accordingly, such an expense may be cost prohibitive for most physicians to own such 3D viewing and rendering software. Furthermore, such an expense would most certainly be cost prohibitive for just about all patients.

Accordingly, an improved ultrasound system and method for overcoming the problems in the art is desired.

According to one embodiment of the present disclosure, a method of providing 3D data sets for stand-alone use includes providing a 3D viewing program on a computer readable media and providing at least one 3D data set on the same computer readable media, wherein responsive to a computer activation of the 3D viewing program, the 3D viewing program operates to provide a 3D rendering based upon the at least one 3D data set.

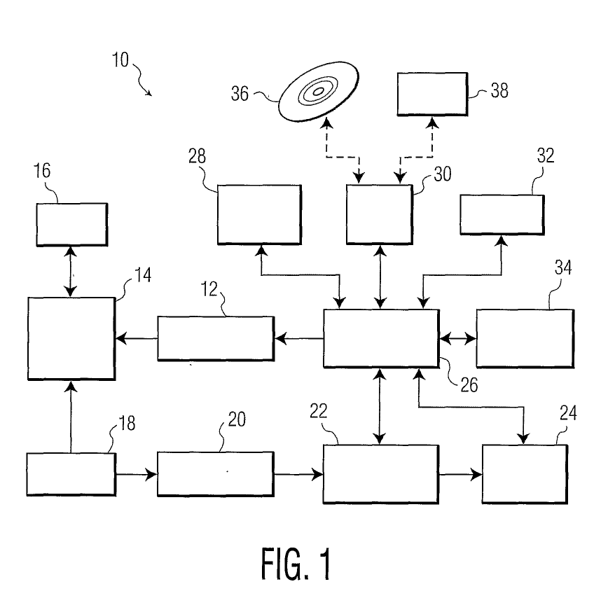

FIG. 1 is a block diagram view of an ultrasound diagnostic imaging system for generating stand-alone 3D ultrasound image data and viewing computer readable media according to one embodiment of the present disclosure;

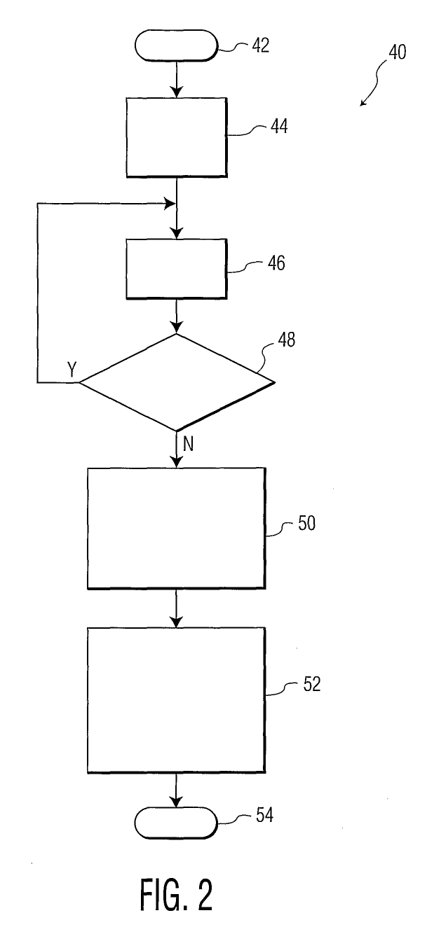

FIG. 2 is a flow diagram view of a method for generating stand-alone 3D ultrasound image data and viewing computer readable media in an ultrasound diagnostic imaging system according to one embodiment of the present disclosure;

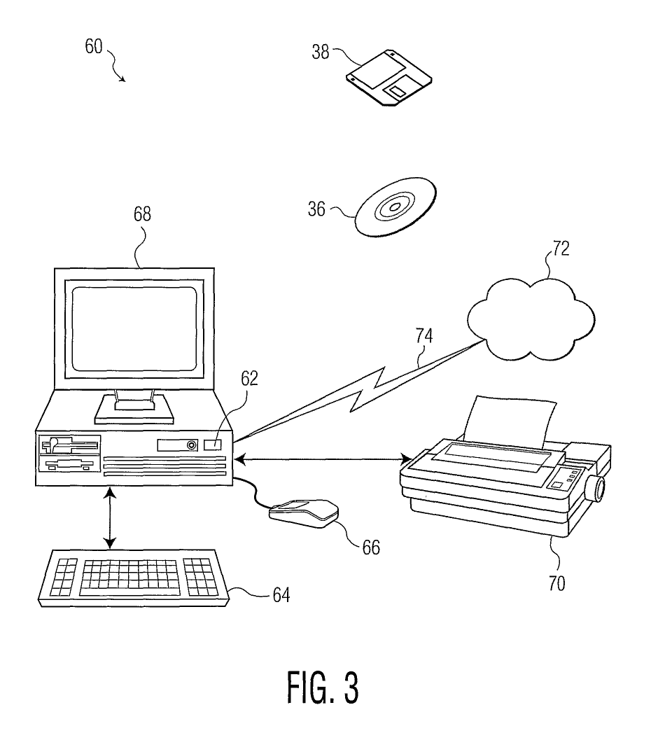

FIG. 3 is a block diagram view of a personal computer for use in accessing the stand-alone 3D ultrasound image data and viewing computer readable media generated by an ultrasound diagnostic imaging system according to one embodiment of the present disclosure;

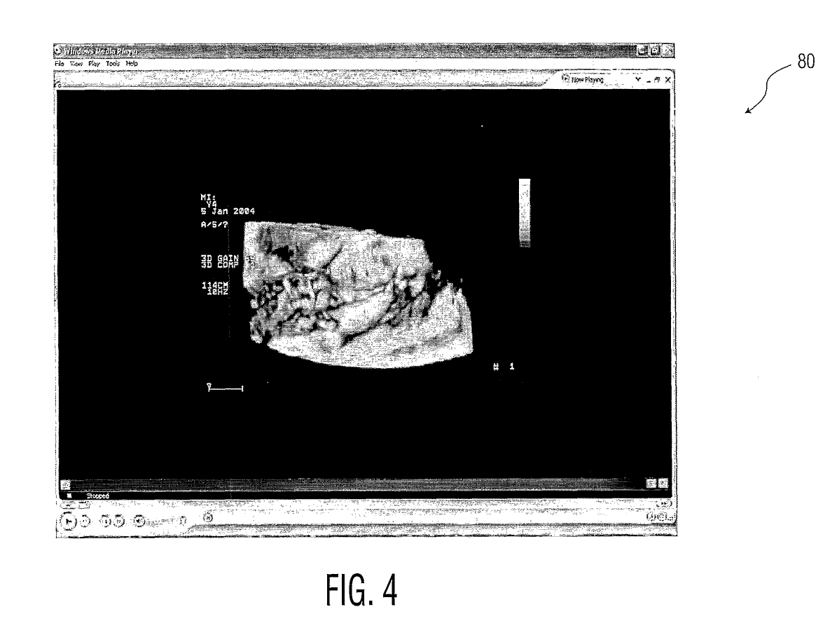

FIG. 4 is an illustrative view of a 3D image obtained from a stand-alone 3D data set and 3D viewing program according to one embodiment of the present disclosure; and

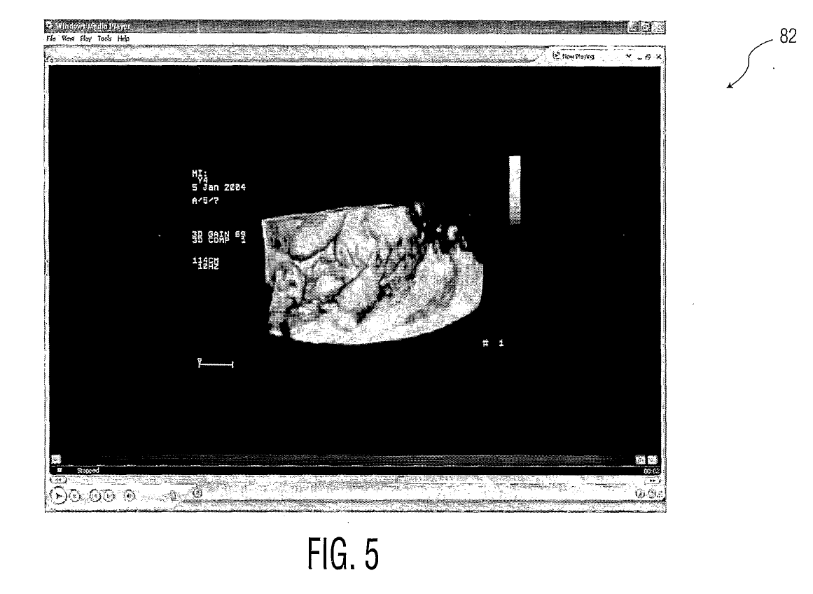

FIG. 5 is an illustrative view of another 3D image obtained from the same stand-alone 3D data set and 3D viewing program of FIG. 4.

The present embodiments relate to storing a simple 3D viewing program on the same compact disc (CD) or other portable personal computer (PC) based media which contains a patient's 3D ultrasound image data set or data sets. In one embodiment, the simple 3D viewing program is designed to run on commercially available computer operating systems, for example, WINDOWS™ available from Microsoft Corporation. Furthermore, the 3D viewing program would rely on a minimal PC configuration. As a result, the 3D viewing program would allow a patient, for example, at home on his/her PC, to review the 3D data at their convenience. Important aspects of the 3D rendering and viewing software include: a) easy to use; and b) small size in terms of memory/storage requirement, as the 3D rendering and viewing software needs to fit on the media, in addition to the 3D data set(s).

Benefits provided by the embodiments of the present disclosure include the following as discussed below. The embodiments of the present disclosure provide a patient with an ability to have a high quality 3D representation of an image captured during an ultrasound exam, which can be rendered in different ways and viewed from multiple angles. This can be especially important to a pregnant woman. That is, the present embodiments provide a way for a pregnant woman to obtain a high quality 3D representation of an image of her baby in utero which can be rendered in different ways and viewed from multiple angles, at the patient's convenience on her PC.

Another benefit of the embodiments of the present disclosure is that a physician can create computer readable media, such as a CD, containing the 3D rendering and viewing software and a patient's 3D data set(s) on the same media, during the patient's office visit. In addition, the physician may be able to charge a nominal fee for the creation of such computer readable media for the patient(s) receiving such computer readable media. Yet another benefit of the embodiments of the present disclosure is that the embodiments would allow a physician to mail or deliver the computer readable media, such as a CD, to a second physician or colleague. As a result, the second physician can review a patient's ultrasound examination information in 3D without the second physician having to have any additional specialized 3D display/review software.

FIG. 1 is a block diagram view of an ultrasound diagnostic imaging system 10 suitable for implementing the various embodiments of the present disclosure. An ultrasound transmitter 12 is coupled through a transmit/receive (T/R) switch 14 to a transducer array or probe 16. In one embodiment, transducer array 16 comprises a two-dimensional array of transducer elements for performing three-dimensional scanning. The transducer array 16 transmits ultrasound energy into a region being imaged and receives reflected ultrasound energy, or echos, from various structures and organs within the patient's body. The transmitter 12 includes a transmit beamformer. By appropriately delaying the pulses applied to each transducer element by transmitter 12, the transmitter transmits a focused ultrasound beam along a desired transmit scan line.

According to one embodiment, array transducer 16 includes a two dimensional array such as disclosed in U.S. Pat. No. 6,428,477, assigned to the assignee of the present disclosure and incorporated herein by reference. U.S. Pat. No. 6,428,477 discloses delivery of therapeutic ultrasound and performing ultrasound diagnostic imaging with the use of a two dimensional ultrasound array. The two dimensional ultrasound array includes a matrix or “grid” of transducer elements. Having a grid or matrix of transducer elements allows three-dimensional (3D) images to be acquired. That is, the matrix of transducer elements makes possible the steering and electronic focusing of ultrasound energy in any arbitrary direction. Unlike the two dimensional ultrasound array, a typical single array of transducer elements allows steering and electronic focusing in only one plane.

The transducer array 16 couples to an ultrasound receiver 18 through T/R switch 14. Reflected ultrasound energy from a given point within the patient's body is received by the transducer elements at different times. The transducer elements convert the received ultrasound energy to received electrical signals which are amplified by receiver 18 and are supplied to a receive beamformer 20. The signals from each transducer element are individually delayed and then are summed by the beamformer 20 to provide a beamformer signal that is a representation of the reflected ultrasound energy level along a given receive scan line. As known in the art, the delays applied to the received signals may be varied during reception of ultrasound energy to effect dynamic focusing. The process is repeated for multiple scan lines to provide signals for generating an image of a region of interest in the patient's body. Because the transducer array is two-dimensional, the receive scan lines can be steered in azimuth and in elevation to form a three-dimensional scan pattern. The beamformer 20 may, for example, be a digital beamformer such as may be found in any suitable commercially available medical diagnostic ultrasound machine.

The beamformer signals are stored in an image data buffer 22 which, as described below, stores image data for different volume segments of an image volume and, in certain instances, for different points of a cardiac cycle. The image data is output from image data buffer 22 to a display system 24 which generates a three-dimensional image of the region of interest from the image data. The display system 24 may include a scan converter which converts sector scan signals from beamformer 20 to conventional raster scan display signals.

A system controller 26 provides overall control of the ultrasound diagnostic imaging system. The system controller 26 performs timing and control functions and typically includes a microprocessor and associated memory. In addition, an ECG device (not shown) can be used, the ECG device including ECG electrodes for being attached to a subject or patient. The ECG device supplies ECG waveforms to system controller 26 for synchronizing imaging to the patient's cardiac cycle.

Ultrasound diagnostic imaging system 10 further includes input element 28, media drive 30, storage 32, and network interface 34, each coupled to system controller 26 for performing functions to be discussed further herein below. Input element 28 can include any suitable input device, for example, a keyboard, mouse, or other suitable input device, for enabling user input to the ultrasound diagnostic imaging system. Media drive 30 includes any suitable media drive, for interfacing with one or more different types of media (36,38). For example, media drive 30 may include an optical read-write drive such as a CD-RW drive. Media drive 30 may also include a read-write disc drive, such as a floppy drive. Still further, media drive 30 may include a drive suitable for reading and writing to a SmartMedia™, CompactFlash™, Memory Stick™, or similar type of storage device.

In addition, storage 32 comprises any suitable computer storage, such as a hard disk drive, for storing computer programs and data as discussed herein with respect to the embodiments of the present disclosure. Furthermore, network interface 34 is coupled to the system controller 26 for enabling system controller 26 to access a network, such as, an intranet, the Internet, an extranet, or other computer network.

In the embodiments of the present disclosure, the computer readable media preferably includes a single computer readable media. For example, media 36 can comprise a recordable CD. Media 38 can comprise, for example, a CompactFlash™ memory card. Furthermore, the computer readable media can include a writable or re-writable CD-ROM or other compact computer readable media. Still further, the computer readable media may include a network communication media. Examples of network communication media include, for example, an intranet, the Internet, or an extranet.

In one embodiment, the 3D viewing program requires a minimal memory/storage on the order of less than 10-25% of the computer readable media. For example, the memory/storage size of the 3D viewing program may be on the order of less than 100M bytes, installed. Furthermore, in one embodiment, the memory requirement is on the order of 20 M bytes, installed. On the other hand, the at least one 3D data set may require a memory/storage allocation on the order of up to more than 75-90% of the computer readable media.

FIG. 2 is a flow diagram view of a method 40 for generating stand-alone 3D ultrasound image viewer and data media using an ultrasound diagnostic imaging system according to one embodiment of the present disclosure. The method 40 begins at step 42 and includes using an ultrasound imaging system for obtaining at least one 3D data set of an ultrasound imaged object. The ultrasound imaging system is provided with a portable version of a 3D image viewing program at step 44. In step 46, a 3D data set is obtained. Subsequent to obtaining the first 3D data set, in step 48, a query includes whether additional data sets are to be obtained. If additional 3D data sets are to be obtained, then the process returns to step 46 for obtaining the additional 3D data set.

Subsequent to obtaining the at least one 3D data set, the 3D viewing program is configured for accessing the at least one 3D data set in step 50. Configuring the 3D viewing program includes editing the 3D viewing program to be able to access and reference the one or more 3D data sets that have been obtained in the steps 46 and 48. In step 52, the one or more 3D data sets and the 3D viewing program are downloaded onto a computer readable media. The process ends at step 54.

Accordingly, the 3D viewing program is responsive to a computer activation of the 3D viewing program for accessing the one or more 3D data sets on the computer readable media. In response to accessing the 3D data set, the 3D viewing program provides 3D rendering data of the ultrasound imaged object as a function of the at least one 3D data set. The 3D rendering data is suitable for use in displaying at least one 3D view of the ultrasound imaged object on a display device. In one embodiment, the 3D viewing program includes one or more of a 3D cropping, 3D rotation, printing, and AVI generation type of 3D viewing program.

FIG. 3 is a block diagram view of a personal computer system 60 for use in accessing the stand-alone 3D ultrasound image data and viewing computer readable media (36,38) generated by an ultrasound diagnostic imaging system according to one embodiment of the present disclosure. Computer system 60 can include any type of information handling system that includes, for example, a computer 62, input device 64 (such as a keyboard), pointing device 66 (such as a mouse), and a display 68. In addition, computer system 60 may include a printer 70 or other type of data presentation/output device. Still further, computer system 60 can be coupled to a network 72, via a suitable network connection 74.

In one embodiment, the computer activation of the stand-alone 3D ultrasound image data and viewing computer readable media (36,38) includes an automatic activation in response to inserting the computer readable media (36,38) into a media drive of the computer 62. In another embodiment, the computer readable media (36,38) is subsequently stored on an end user computer as a 3D ultrasound image data set and viewer program file. In the later embodiment, computer activation includes an activation in response to a user initiation corresponding to a selection of the 3D ultrasound image data set and viewer program file.

In one example, responsive to a user input via the input device 64, the computer 62 accesses the stand-alone 3D data sets and 3D viewing program media for causing the display 68 to render a 3D view of the at least one 3D data set as a function of the user input. User input can include, for example, selecting one or more of 3D cropping of a 3D view, a 3D rotation of the 3D view, printing a 3D view, or initiating an AVI generation of the 3D view.

FIG. 4 is an illustrative view of a 3D image 80 obtained from a stand-alone 3D data set and 3D viewing program according to one embodiment of the present disclosure. FIG. 5 is an illustrative view of another 3D image 82 obtained from the same stand-alone 3D data set and 3D viewing program of FIG. 4. In the example shown, the 3D viewing program generates AVI type files for display on the display device, using for example, a Media Player from Microsoft Corporation. This is merely one example, and other functionalities are possible.

According to another embodiment, the 3D viewing program can be pre-loaded onto the computer readable media prior to downloading and/or saving the at least one 3D data set onto the computer readable media. In yet another embodiment, the at least one 3D data set and the 3D viewing program can be downloaded onto (or saved to) the computer readable media by saving or recording the at least one 3D data set onto the computer readable media prior to saving or recording the 3D viewing program onto the computer readable media.

According to yet another embodiment, a method for generating 3D ultrasound image data set and viewer media of an ultrasound imaged object includes providing a 3D viewing program and at least one 3D data set on the same computer readable media. Responsive to a computer activation of the 3D viewing program, the 3D viewing program accesses the at least one 3D data set and provides 3D rendering data. The 3D rendering data is suitable for generation of a 3D view of the ultrasound imaged object on a display. The 3D view is a function of the at least one 3D data set. The 3D ultrasound image data set and viewer media is designed for stand-alone use on a computer, for example, a doctor's or a patient's personal computer.

Still another embodiment of the present disclosure includes a method for generating stand-alone 3D ultrasound image data and viewing computer readable media. The method includes providing a 3D viewing program on a computer readable media and obtaining at least one 3D data set of an ultrasound imaged object using an ultrasound imaging system. The method further includes providing the at least one 3D data set on the same computer readable media, wherein the 3D viewing program is configured to operate in response to a computer activation of the 3D viewing program. Responsive to the computer activation, the 3D viewing program accesses the at least one 3D data set on the computer readable media and provides 3D rendering data of the ultrasound imaged object based upon the at least one 3D data set. The 3D rendering data is configured for use in displaying at least one 3D view of the ultrasound imaged object on a display device. In other words, the display device displays the at least one 3D view in response to the display device receiving the 3D rendering data.

In another embodiment, an ultrasound imaging system includes an ultrasound imaging device for obtaining at least one 3D data set of an ultrasound imaged object. The ultrasound imaging system further includes a media device for providing a 3D ultrasound image viewing program on a computer readable media. The media device is further for providing the at least one 3D data set of the ultrasound imaged object on the same computer readable media. Responsive to a computer activation of the 3D viewing program, for example, on a doctor's or patient's personal computer, the 3D viewing program accesses the at least one 3D data set and provides 3D rendering data suitable for generation of a 3D view of the ultrasound imaged object on a display. The 3D view is a function of the at least one 3D data set. Accordingly, the computer readable media comprises 3D ultrasound image data set and viewer media of the ultrasound imaged object. In one embodiment, the 3D ultrasound image data set and viewer media is for stand-alone use on a computer.

In yet another embodiment, an ultrasound imaging system comprises an ultrasound imaging device for obtaining at least one 3D data set of an ultrasound imaged object and a device for providing a portable 3D image viewing program. The ultrasound imaging system further includes a device for storing and/or recording the at least one 3D data set and the 3D viewing program onto a computer readable media. In one embodiment, the computer readable media comprises a stand-alone 3D ultrasound image viewer and data media. The 3D viewing program is responsive to a computer activation of the 3D viewing program for accessing the at least one 3D data set and for providing 3D rendering data of the ultrasound imaged object as a function of the at least one 3D data set. The 3D rendering data is suitable for use in displaying at least one 3D view of the ultrasound imaged object on a display device.

The device for providing the 3D image viewing program includes any suitable device known in the art, for example, one or more of a storage device, a media drive device, or a network interface device. In addition, the device for storing and/or recording the at least one 3D data set and the 3D image viewing program onto a computer readable media includes any suitable device known in the art, for example, one or more of a storage device, a media drive device, and a network interface device.

In one embodiment, the computer readable media includes a single computer readable media. The computer readable media can further include a writable or re-writable CD-ROM. Still further, the computer readable media can include a compact disc media. Yet further, the computer readable media can include a network communication media, wherein the network communication media includes an intranet, Internet, or extranet.

In a still further embodiment, an ultrasound imaging system including a feature for generating stand-alone 3D ultrasound image data and viewing computer readable media comprises an ultrasound imaging device and a media recording device. The ultrasound imaging device obtains at least one 3D data set of an ultrasound imaged object. The media recording device provides a 3D viewing program on a computer readable media and provides the at least one 3D data set on the same computer readable media. The 3D viewing program is configured to operate in response to a computer activation of the 3D viewing program for accessing the at least one 3D data set and providing 3D rendering data of the ultrasound imaged object based upon the at least one 3D data set. Furthermore, the 3D rendering data is configured for use in displaying at least one 3D view of the ultrasound imaged object on a display device.

Additional aspects of the present embodiments include the following. In one embodiment, the computer readable media containing the 3D viewing program and the 3D data set(s) are appropriately designed to operate on any computer, wherein different computers may have different operating systems, for example, a Microsoft Windows™ based operating system, an Apple Mac™ based operating system, or other operating system. In another embodiment, the 3D viewing program and 3D data set(s) are not solely restricted to just media, for example, the 3D viewing program and 3D data set(s) may be encapsulated in a compressed file, such as a zip file. As a result, the zip file containing the 3D viewing program and 3D data set(s) could then be transmitted via email to a patient and/or colleague.

In yet another embodiment, the 3D viewing program is encapsulated in the 3D data set(s). In addition, the 3D data set(s) with the encapsulated 3D viewing program is made into an executable file. Accordingly, a patient or colleague need only “click” on the corresponding file to initiate 3D viewing of the 3D data set(s). In yet a further embodiment, the 3D viewing program executes automatically in response to the computer readable media being inserted within a media drive of the computer being used by the patient or colleague.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

1. A method for generating 3D ultrasound image data set and viewer media of an ultrasound imaged object, comprising:
providing a 3D viewing program (44) on a computer readable media (36); and
providing at least one 3D data set (46) on the same computer readable media, wherein responsive to a computer activation of the 3D viewing program, the 3D viewing program accesses the at least one 3D data set and provides 3D rendering data suitable for generation of a 3D view of the ultrasound imaged object on a display (24,68), the 3D view being a function of the at least one 3D data set.

2. The method of claim 1, further wherein the 3D ultrasound image data set and viewer media (52) is for stand-alone use on a computer.

3. A method for producing stand-alone 3D ultrasound image viewer and data media, comprising:
using an ultrasound imaging system (10) for obtaining at least one 3D data set of an ultrasound imaged object;
providing a 3D image viewing program (44); and
providing the at least one 3D data set (44) and the 3D viewing program onto a computer readable media (36), wherein the 3D viewing program is responsive to a computer activation of the 3D viewing program for accessing the at least one 3D data set and for providing 3D rendering data of the ultrasound imaged object as a function of the at least one 3D data set, the 3D rendering data being suitable for use in displaying at least one 3D view of the ultrasound imaged object on a display device (24,68).

4. The method of claim 3, wherein the computer readable media (36,38,72) includes one selected from the group consisting of a single computer readable media, a writable or re-writable CD-ROM, a compact disc media, and a network communication media.

5. The method of claim 4, wherein the network communication media (72) includes an intranet, Internet, or extranet.

6. The method of claim 3, wherein providing the 3D viewing program includes pre-loading the 3D viewing program onto the computer readable media.

7. The method of claim 3, wherein providing the at least one 3D data set and the 3D viewing program onto the computer readable media includes recording the at least one 3D data set onto the computer readable media prior to recording the 3D viewing program onto the computer readable media.

8. The method of claim 3, wherein the 3D viewing program includes at least one selected from the group consisting of 3D cropping, 3D rotation, printing, and AVI generation.

9. A method for generating stand-alone 3D ultrasound image data and viewing computer readable media, comprising:
providing a 3D viewing program on a computer readable media (52);
obtaining at least one 3D data set of an ultrasound imaged object using an ultrasound imaging system (46); and
providing the at least one 3D data set on the computer readable media, wherein the 3D viewing program is configured to operate in response to a computer activation of the 3D viewing program for accessing the at least one 3D data set and providing 3D rendering data of the ultrasound imaged object based upon the at least one 3D data set, wherein the 3D rendering data is configured for use in displaying at least one 3D view of the ultrasound imaged object on a display device (24,68).

10. The method of claim 9, further comprising:
displaying the at least one 3D view on the display device in response to the display device receiving the 3D rendering data.

11. The method of claim 9, wherein the 3D viewing program includes at least one selected from the group consisting of 3D cropping, 3D rotation, printing, and AVI generation.

12. The method of claim 9, wherein the 3D viewing program requires a minimal portion of the computer readable media, on the order of less than 100 Mbytes.

13. The method of claim 9, wherein the 3D viewing program requires an allocation on the order of less than 10-25% of the computer readable media and the at least one 3D data set requires a memory/storage allocation on the order of 75-90% or more.

14. The method of claim 9, wherein the computer readable media (36,38,72) includes one selected from the group consisting of a writable or re-writable CD-ROM, a compact disc media, and a network communication media.

15. The method of claim 14, wherein the network communication media (72) includes an intranet, Internet, or extranet.

16. An ultrasound imaging system, comprising:
an ultrasound imaging device (10) for obtaining at least one 3D data set of an ultrasound imaged object; and
a media device (30) for providing a 3D ultrasound image viewing program on a computer readable media (36) and for providing the at least one 3D data set of the ultrasound imaged object on the same computer readable media, wherein responsive to a computer activation of the 3D viewing program, the 3D viewing program accesses the at least one 3D data set and provides 3D rendering data suitable for generation of a 3D view of the ultrasound imaged object on a display (24), the 3D view being a function of the at least one 3D data set, wherein the computer readable media includes 3D ultrasound image data set and viewer media of the ultrasound imaged object.

17. The ultrasound imaging system of claim 16, further wherein the 3D ultrasound image data set and viewer media is for stand-alone use on a computer.

18. An ultrasound imaging system comprising:
an ultrasound imaging device (10) for obtaining at least one 3D data set of an ultrasound imaged object;
a device (32) for providing a 3D image viewing program; and
a device (30) for recording the at least one 3D data set and the 3D viewing program onto a computer readable media, wherein the computer readable media includes a stand-alone 3D ultrasound image viewer and data media and wherein the 3D viewing program is responsive to a computer activation of the 3D viewing program for accessing the at least one 3D data set and for providing 3D rendering data of the ultrasound imaged object as a function of the at least one 3D data set, the 3D rendering data being suitable for use in displaying at least one 3D view of the ultrasound imaged object on a display device (24).

19. The ultrasound imaging system of claim 18, wherein the device (32) for providing the 3D image viewing program includes one selected from the group consisting of a storage device (32), a media drive device (30), and a network interface device (34).

20. The ultrasound imaging system of claim 18, wherein the device for recording the at least one 3D data set and the 3D image viewing program onto a computer readable media includes one selected from the group consisting of a storage device (32), a media drive device (30), and a network interface device (34).

21. The ultrasound imaging system of claim 18, wherein the computer readable media (36,38,72) includes one selected from the group consisting of a single computer readable media, a writable or re-writable CD-ROM, a compact disc media, and a network communication media.

22. The ultrasound imaging system of claim 21, wherein the network communication media (72) includes an intranet, Internet, or extranet.

23. The ultrasound imaging system of claim 18, wherein providing the 3D viewing program includes pre-loading the 3D viewing program onto the computer readable media.

24. The ultrasound imaging system of claim 18, wherein providing the at least one 3D data set and the 3D viewing program onto the computer readable media includes recording the at least one 3D data set onto the computer readable media prior to recording the 3D viewing program onto the computer readable media.

25. The ultrasound imaging system of claim 18, wherein the 3D viewing program includes at least one selected from the group consisting of 3D cropping, 3D rotation, printing, and AVI generation.

26. An ultrasound imaging system including a feature for generating stand-alone 3D ultrasound image data and viewing computer readable media, said ultrasound imaging system comprising:
an ultrasound imaging device (16) for obtaining at least one 3D data set of an ultrasound imaged object; and
a media recording device (30) for providing a 3D viewing program on a computer readable media and for providing the at least one 3D data set on the same computer readable media, wherein the 3D viewing program is configured to operate in response to a computer activation of the 3D viewing program for accessing the at least one 3D data set and providing 3D rendering data of the ultrasound imaged object based upon the at least one 3D data set, wherein the 3D rendering data is configured for use in displaying at least one 3D view of the ultrasound imaged object on a display device (24).

27. The ultrasound imaging system of claim 26, wherein the computer activation includes an automatic activation in response to inserting the computer readable media into a media drive of the computer.

28. The ultrasound imaging system of claim 26, wherein the computer readable media is subsequently stored on an end user computer as a 3D ultrasound image data set and viewer program file, and wherein the computer activation includes an activation in response to a user initiation corresponding to a selection of the 3D ultrasound image data set and viewer program file.
